(19) United States
(12) Patent Application Publication
(10) Pub. No.: US 2013/0144097 A1
(43) Pub. Date: Jun. 6, 2013

(54) Aromatics Production Process and Apparatus

(71) Applicants: Timothy P. Bender, Houston, TX (US); John W. Rebeck, Katy, TX (US); Rimas V. Vebeliunas, Houston, TX (US); John R. Porter, Friendswood, TX (US); Anthony Go, Houston, TX (US); Larry L. Iaccino, Seabrook, TX (US); Glenn C. Wood, Houston, TX (US)

(72) Inventors: Timothy P. Bender, Houston, TX (US); John W. Rebeck, Katy, TX (US); Rimas V. Vebeliunas, Houston, TX (US); John R. Porter, Friendswood, TX (US); Anthony Go, Houston, TX (US); Larry L. Iaccino, Seabrook, TX (US); Glenn C. Wood, Houston, TX (US)

(73) Assignee: ExxonMobil Chemical Patents Inc., Baytown, TX (US)

(21) Appl. No.: 13/676,813

(22) Filed: Nov. 14, 2012

Related U.S. Application Data
(60) Provisional application No. 61/567,169, filed on Dec. 6, 2011.

(51) Int. Cl. C07C 2/64; C07C 5/22; B01J 19/00

(52) U.S. Cl. CPC C07C 2/64; B01J 19/0046; C07C 5/22
USPC 585/254; 422/187; 585/304

(57) ABSTRACT

In a process for producing para-xylene, a naphtha feed is reformed under conditions effective to convert at least 50 wt % of the naphthenes in the naphtha feed to aromatics, but to convert no more than 25 wt % of the paraffins in the naphtha feed, and thereby produce a reforming effluent. A first stream containing benzene and/or toluene is removed from the reforming effluent and is fed to a xylene production unit under conditions effective to convert benzene and/or toluene to xylenes. In addition, a second stream containing C8 aromatics is removed from the reforming effluent and is fed, together with at least part of the xylenes produced in the xylene production unit, to a para-xylene recovery unit to recover a para-xylene product stream and leave a para-xylene-depleted C8 stream. At least part of para-xylene-depleted C8 stream is then fed to a xylene isomerization unit effective to isomerize xylenes in para-xylene-depleted stream back towards an equilibrium mixture of xylenes and thereby produce an isomerization effluent. The isomerization effluent is then recycled to the para-xylene extraction unit.

### PRIORITY CLAIM

This application claims the benefit of Provisional Application No. 61/567,169, filed Dec. 6, 2011, the disclosure of which is incorporated by reference in its entirety.

### FIELD

This invention relates to a process and apparatus for the production of aromatic hydrocarbons.

### BACKGROUND

Benzene, toluene, and xylenes (BTX) are important aromatic hydrocarbons, for which the worldwide demand is steadily increasing. The demand for xylenes, particularly para-xylene, has increased in proportion to the increase in demand for polyester fibers and film. Benzene is a highly valuable product for use as a chemical raw material. Toluene is also a valuable petrochemical for use as a solvent and an intermediate in chemical manufacturing processes and as a high octane gasoline component.

A major source of benzene, toluene, and xylenes (BTX) is catalytic reformate, which is produced by contacting petroleum naphtha with a hydrogenation/dehydrogenation catalyst on a support. The resulting reformate is a complex mixture of paraffins, the desired C6 to C8 aromatics, in addition to a significant quantity of heavier aromatic hydrocarbons. Usually, a C6 to C₈ fraction is separated from the reformate, extracted with a solvent selective for aromatics or aliphatics to produce a mixture of aromatic compounds that is relatively free of aliphatics. This mixture of aromatic compounds is composed of benzene, toluene and xylenes (BTX), along with ethyl benzene.

Conventional aromatics plants are designed and operated to maximize the yield of the desired aromatics products, notably para-xylene, from the naphtha feed. This typically involves topping the naphtha feed to remove iso-hexane and lighter molecules, which tend to have low aromatics yield in the reformer, and then feeding the remaining heavy virgin naphtha (HVN) to a high severity continuous catalytic reformer. By employing a reforming catalyst comprising a strong hydrogenation/dehydrogenation metal on an acidic support, such as a halogen-treated alumina, aromatics make is maximized. However, this is accompanied by significant hydrogen production and cracking of the naphtha to light gas (C4−) products. In addition, xylenes make is normally increased by recovering at least the C9+ aromatics from the heavy ends of the reformate and feeding the C9+ aromatics to a transalkylation unit with part of the benzene and/or toluene produced in the reformer. Additionally, to increase yields, the transalkylation catalyst normally includes a dealkylation component which dealkylates ethyl and propyl groups from the C9+ aromatics feed to produce benzene and methylated benzenes as well as light gas products.

The xylene produced in a conventional aromatics plant is normally fed to a para-xylene recovery unit, typically a fractional crystallization unit or para-selective adsorption process (e.g., Parex or Eluxyl). The para-xylene depleted raffinate from this unit is then fed to an isomerization unit, which isomerizes the xylenes back to an equilibrium mixture and converts the ethylbenzene entrained in the xylene product. Normally, the xylene isomerization is chosen so as to convert the ethylbenzene by cracking to benzene and ethane since this requires less energy and capital investment than a catalyst that converts the ethylbenzene by isomerization to additional xylenes.

The plant configuration and operation described above is economically attractive in many regions of the world, where the co-produced hydrogen and light gases have significant value. However, in other regions of the world, such as the Middle East, the co-production of hydrogen and light gases represents a significant downgrade to process economics, since these products either have little utility or little value. There is therefore a need for a process and apparatus for producing aromatics, and especially para-xylene, that minimizes the co-production of hydrogen and light gases.

### SUMMARY

In one aspect, the invention resides in a process for producing para-xylene, the process comprising:

(a) reforming a naphtha feed under reforming conditions effective to convert at least 50 wt %, such at least 75 wt %, of the naphthenes in the naphtha feed to aromatics, but to convert no more than 25 wt %, such as no more than 10 wt %, of the paraffins in the naphtha feed, and thereby produce a reforming effluent;

(b) removing at least a first stream containing benzene and/or toluene and a second stream containing C8 aromatics from the reforming effluent; (c) feeding at least part of the benzene and/or toluene from the first stream to a xylene production unit under conditions effective to convert benzene and/or toluene to xylenes; (d) feeding at least part of the C8 aromatics from the second stream and at least part of the xylenes produced in (c) to a para-xylene recovery unit to recover a para-xylene product stream and leave a para-xylene-depleted C8 stream;

(e) feeding at least part of para-xylene-depleted C8 stream to a xylene isomerization unit effective to isomerize xylenes in said stream back towards an equilibrium mixture of xylenes and thereby produce an isomerization effluent; and

(f) recycling the isomerization effluent to the para-xylene extraction unit.

Conveniently, at least 25 wt %, such as at least 35 wt %, for example at least 80 wt % of the naphtha feed comprises C7 and C8 hydrocarbons.

In one embodiment, the reforming (a) is conducted in one or more fixed bed reforming units, typically using a catalyst comprising platinum and rhenium.

In another embodiment, the reforming (a) is conducted in one or more moving bed reforming units, typically using a catalyst comprising platinum and tin.

Conveniently, said removing (b) comprises a solvent extraction and/or extractive distillation to separate the reforming effluent into an aromatics fraction and a non-aromatics fraction.

In one embodiment, the process further comprises separating benzene from said aromatics fraction and optionally reacting at least part of the separated benzene with hydrogen produced by said reforming (a) to convert said benzene to cyclohexane.

Conveniently, the xylene production unit effects disproportionation of toluene to produce benzene and xylenes.

Alternatively, said xylene production unit effects alkylation of benzene and/or toluene with methanol to produce xylenes.

In one embodiment, the xylene isomerization unit is effective to convert ethylbenzene in said para-xylene-depleted C8 stream to xylenes.

Conveniently, no more than 10 wt %, such as less than 2 wt %, of said feed is converted to hydrocarbons having 4 or less carbon atoms in steps (a), (c) and (e) combined.

In a further aspect, the invention resides in a para-xylene production plant comprising:

(a) a first separation system for removing C6− hydrocarbons and C9+ hydrocarbons from a C₅ to C₁₂ hydrocarbon fraction to produce a naphtha feed;

(b) at least one reforming unit for converting at least 50 wt % of the naphthenes in the naphtha feed to aromatics, but to convert no more than 25 wt % of the paraffins in the naphtha feed, and thereby produce a reforming effluent;

(c) a second separation system for separating the reforming effluent into an aromatics fraction and a non-aromatics fraction;

(d) a third separation system for separating the aromatics fraction into a first stream containing benzene and/or toluene and a second C8 aromatic-containing stream;

(e) a xylene production unit for converting at least part of the benzene and/or toluene in the first stream to xylenes; and

(f) a fourth separation system for selectively recovering para-xylene from the second C8 aromatic-containing stream and the xylenes produced in said xylene production unit to leave a para-xylene-depleted C8 stream;

(g) a xylene isomerization unit effective to isomerize xylenes in said para-xylene-depleted C8 stream back towards an equilibrium mixture of xylenes and to isomerize ethylbenzene in said stream to xylenes and thereby produce an isomerization effluent; and

(h) means for recycling said isomerization effluent to the fourth separation system.

### BRIEF DESCRIPTION OF THE DRAWINGS

FIG. 1 is a schematic diagram of a process for producing para-xylene according to one embodiment of the invention.

### DETAILED DESCRIPTION OF THE EMBODIMENTS

Described herein is a process and apparatus for producing para-xylene which are configured to minimize the amount of hydrogen and light gases co-produced with the desired para-xylene. Thus, in the present process, a naphtha feed is reformed under mild reforming conditions effective to convert at least 50 wt %, such as at least 75 wt %, even at least 99 wt %, of the naphthenes in the naphtha feed to aromatics, but to convert no more than 25 wt %, such as no more than 10 wt %, even no more than 1 wt %, of the paraffins in the naphtha feed. The resulting reformate is then separated into at least a C8 aromatics stream and a toluene stream. At least part of the toluene stream is fed to a toluene production unit to convert the toluene to xylenes, while at least part of the C8 aromatics stream and at least part of the xylenes produced in toluene production unit are fed to a para-xylene recovery unit to recover a para-xylene product stream and leave a para-xylene-depleted C8 stream. At least part of para-xylene-depleted C8 stream is then fed to a xylene isomerization unit effective to isomerize xylenes in said stream back towards an equilibrium mixture of xylenes thereby producing an isomerization effluent. The isomerization effluent is then recycled to the para-xylene extraction unit.

As used herein the term “naphthenes” is used to mean saturated cyclic hydrocarbons having a 5 or 6 carbon member ring (for example, alkylcyclopentanes, cyclohexane, and alkylcyclohexanes) while the term “paraffins” is used to mean saturated non-cyclic hydrocarbons.

### Naphtha Feed

Any naphtha feed conventionally used as a reformer feedstock can be employed in the reforming stage of the present process. Typically the feed to the reforming stage is composed of a C₅ to C₁₂ hydrocarbon fraction of which at least 25 wt %, generally about 31 to about 46 wt %, comprises C7 and C8 hydrocarbons. More preferably, the feed is fractionated to remove C6− hydrocarbons before being supplied to the reforming stage and comprises at least 35 wt %, such as generally about 37 to about 54 wt %, C7 and C8 hydrocarbons. Even more preferably, the feed is fractionated to remove C6− hydrocarbons and C9+ hydrocarbons before being supplied to the reforming stage and comprises at least 80 wt %, such as at least 87 wt %, C7 and C8 hydrocarbons.

### Naphtha Reforming

After fractionation as necessary to bring the naphtha feed within the compositional range described above, which can be determined by one of ordinary skill in the art in possession of the present disclosure, such as by routine experimentation to determine the appropriate boiling ranges, the feed is supplied to a low severity reformer typically in the form of one, or more preferably, a plurality of reforming units each containing a fixed or moving bed of reforming catalyst. Generally, the reforming catalyst comprises at least one, and generally a plurality of, hydrogenation/dehydrogenation metals on an inorganic oxide support. Suitable hydrogenation/dehydrogenation metals include platinum, tin, iridium and rhenium, whereas suitable supports include alumina, silica and silica/alumina. For example, a reforming catalyst useful in the present process typically comprises 0.01 to 2 wt %, such as from 0.1 to 0.7 wt %, of platinum; 0.01 to 2 wt %, such as 0.02 wt % to about 0.4 wt %, of tin and up to 2 wt %, such as from 0.1 to 0.7 wt %, of iridium and/or rhenium on an alumina or chlorided alumina support.

Reforming according to the present invention is generally conducted under conditions including a temperature of about 400° C. to about 600° C., such as about 460° C. to about 540° C., a pressure of about 50 psig to about 750 psig (445 to 5272 kPa), such as about 100 to about 300 psig (790 to 2170 kPa), and a hydrocarbon weight hourly space velocity of about 0.25 to about 4, such as about 1 to about 3.

In particular, the reforming conditions are controlled so as to convert at least 50 wt %, preferably at least 50 wt %, such as at least 75 wt %, for example at least 99 wt %, of the naphthenes in the feed to aromatics, while at the same time converting no more than 25 wt %, such as no more than 10 wt %, for example no more than 1 wt %, of the paraffins in the feed and thereby produce a reforming effluent in which liquid volume yield is maximized and gas yield minimized.

The product of the low severity reforming operation comprises an aromatic-enriched C6+ hydrocarbon stream together with hydrogen and some C₅− fuel gas. After removal of the hydrogen and fuel gas, the reformate stream is directed to an aromatics extraction unit either directly or after initial passage through a heavy aromatics splitter for removal of C9+ hydrocarbons from the reformate. In the aromatics extraction unit, the reformate is subjected to solvent extraction and/or extractive distillation to separate the reformate effluent into an aromatics fraction and a non-aromatics fraction. The aromatics fraction is then fractionated to produce a benzene and toluene-containing stream, at least part of which is passed to a xylene production unit, and C8 aromatic-containing stream, at least part of which is passed to a xylene isomerization and recovery section.

### Xylene Production

Xylene production in the present process can be effected either by toluene disproportionation or by methylation of benzene and/or toluene with methanol. In both cases, the processes are preferably operated so to selectively produce para-xylene over the other xylene isomers.

Where xylene production is effected by toluene disproportionation, the benzene and toluene-containing stream removed from the aromatic fraction of the reformate is initially passed to a benzene fractionation column where most of the benzene is removed to leave a toluene-rich remaining stream, which is then passed to a toluene disproportionation unit. In the toluene disproportionation unit, the toluene-rich stream is contacted with hydrogen in the presence of a zeolite catalyst under disproportionation conditions including a reactor inlet temperature of from about 200° C. to about 500° C., preferably from 350° C. to about 500° C.; a pressure of from about atmospheric to about 5000 psia (100 to 34475 kPa), preferably from about 100 to about 1000 psia (690 to 6900 kPa); a WHSV of from about 0.1 to about 20, preferably from about 2 to about 10; and a H₂/HC mole ratio of from about 0.1 to about 20, preferably from about 1 to about 10.

The zeolite catalyst employed in the toluene disproportionation unit is typically ZSM-5 having a silica to alumina less than 60, such as from 20 to 40, and a crystal size greater than 0.1 micron, such as from 0.1 to 1 micron, for example from 0.1 to 0.5 micron. The zeolite is typically combined with a support or binder material (binder), preferably an inert, non-alumina containing material, such as a porous inorganic oxide support or a clay binder. One such preferred inorganic oxide is silica. Other examples of such binder materials include, but are not limited to, zirconia, magnesia, titania, thoria and boria. These materials may be utilized in the form of a dried inorganic oxide gel or as a gelatinous precipitate. Suitable examples of clay binder materials include, but are not limited to, bentonite and kieselguhr. The relative proportion of catalyst to binder material is generally from about 30 wt % to about 98 wt %, such as from about 50 wt % to about 80 wt %.

The catalyst may be further modified in order to reduce the amount of undesirable by-products, particularly ethylbenzene, produced in the toluene disproportionation process. Such modification typically involves incorporating a hydrogenation/dehydrogenation function within the catalyst, such as by addition of a metal compound such as platinum. While platinum is the preferred metal, other metals of Groups IB to VIII of the Periodic Table such as palladium, nickel, copper, cobalt, molybdenum, rhodium, ruthenium, silver, gold, mercury, osmium, iron, zinc, cadmium, and mixtures thereof, may be utilized. The metal may be added by cation exchange, in amounts of from about 0.001 wt % to about 2 wt %, typically about 0.5 wt %. For example, a platinum modified catalyst can be prepared by first adding the catalyst to a solution of ammonium nitrate in order to convert the catalyst to the ammonium form. The catalyst is subsequently contacted with an aqueous solution of tetraamine platinum(II) nitrate or tetraamine platinum(II) chloride. The catalyst can then be filtered, washed with water and calcined at temperatures of from about 250° C. to about 500° C.

In order to increase its selectivity for the production of para-xylene, the catalyst employed in the toluene disproportionation process is normally subjected to multiple stages of silicon selectivation. Each silicon selectivation stage involves impregnating the catalyst with a silicon compound, normally an organosilicon compound, in a carrier liquid, followed by one or more calcination steps to remove the carrier liquid and convert the organosilicon compound to silica.

Useful selectivating agents include siloxanes which can be characterized by the general formula:

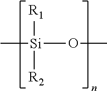

where R₁ is hydrogen, halogen, hydroxyl, alkyl, halogenated alkyl, aryl, halogenated aryl, aralkyl, halogenated aralkyl, alkaryl or halogenated alkaryl. The hydrocarbon substituents generally contain from 1 to 10 carbon atoms, preferably methyl, ethyl, or phenyl groups. R₂ is independently selected from the same group as R₁, and n is an integer of at least 2 and generally in the range of 3 to 1000. The molecular weight of the silicone compound employed is generally between about 80 and about 20,000 and preferably within the approximate range of 150 to 10,000. Representative silicone compounds include dimethyl silicone, diethyl silicone, phenylmethyl silicone, methylhydrogen-silicone, ethylhydrogen silicone, phenylhydrogen silicone, methylethyl silicone, phenylethyl silicone, diphenyl silicone, methyltrifluoropropyl silicone, ethyltri-fluoropropyl silicone, polydimethyl silicone, tetrachloro-phenylmethyl silicone, tetrachlorophenylethyl silicone, tetrachlorophenylhydrogen silicone, tetrachlorophenylphenyl silicone, methylvinyl silicone and ethylvinyl silicone. The silicone compound need not be linear, but may be cyclic, for example, hexamethyl cyclotrisiloxane, octamethyl cyclo-tetrasiloxane, hexaphenyl cyclotrisiloxane and octaphenyl cyclotetrasiloxane. Mixtures of these compounds may also be used, as may silicones with other functional groups.

Preferably, the kinetic diameter of the para-selectivating agent is larger than the zeolite pore diameter, in order to avoid entry of the selectivating agent into the pore and any concomitant reduction in the internal activity of the catalyst. Preferred silicon-containing selectivating agents include dimethylphenylmethyl polysiloxane (e.g., Dow-550), and phenylmethyl polysiloxane (e.g., Dow-710). Dow-550 and Dow-710 are available from Dow Chemical Co., Midland, Mich.

Examples of suitable organic carriers for the selectivating silicon compound include hydrocarbons such as linear, branched, and cyclic alkanes having five or more carbons. In the methods of the present invention it is preferred that the carrier be a linear, branched, or cyclic alkane having a boiling point greater than about 70° C., and most preferably containing 6 or more carbons. Optionally, mixtures of low volatility organic compounds, such as hydrocracker recycle oil, may also be employed as carriers. Particular low volatility hydrocarbon carriers of selectivating agents are decane and dodecane.

The products of the toluene disproportionation process are benzene and a para-xylene-rich mixture of xylenes together with unreacted toluene, small quantities of light gas and heavy C9+ by-products. The product effluent is therefore initially passed to one or more fractionators where the C7− components and the C9+ by-products are removed before the remaining C8 mixture is fed to the xylene isomerization and recovery section described in more detail below.

Where xylene production is effected by alkylation with methanol, the entire benzene and toluene-containing stream removed from the aromatic fraction of the reformate can be fed to the alkylation step, or alternatively part or all of the benzene can be removed from the aromatic fraction so that a toluene-rich fraction is fed to the alkylation step. In either case, alkylation is conducted by contacting benzene and/or toluene with methanol in the presence of a specific zeolite catalyst at a temperature between about 500 and about 700° C., preferably between about 500 and about 600° C., a pressure of between about 1 atmosphere and 1000 psig (100 and 7000 kPa), a weight hourly space velocity of between about 0.5 and 1000, and a molar ratio of toluene to methanol (in the reactor charge) of at least about 0.2, e.g., from about 0.2 to about 20. The process is preferably conducted in the presence of added hydrogen and/or added water such that the molar ratio of hydrogen and/or water to benzene/toluene+ methanol in the feed is between about 0.01 and about 10.

The zeolite catalyst employed in the alkylation process is selected to have a Diffusion Parameter for 2,2-dimethylbutane of about 0.1-15 sec⁻¹, and preferably 0.5-10 sec⁻¹, when measured at a temperature of 120° C. and a 2,2-dimethylbutane pressure of 60 torr (8 kPa). As used herein, the Diffusion Parameter of a particular porous crystalline material is defined as D/r²×10⁶, wherein D is the diffusion coefficient (cm²/sec) and r is the crystal radius (cm). The required diffusion parameters can be derived from sorption measurements provided the assumption is made that the plane sheet model describes the diffusion process. Thus for a given sorbate loading Q, the value Q/Q_{∞}, where Q_{∞} is the equilibrium sorbate loading, is mathematically related to (Dt/r²)^{1/2} where t is the time (sec) required to reach the sorbate loading Q. Graphical solutions for the plane sheet model are given by J. Crank in “The Mathematics of Diffusion”, Oxford University Press, Ely House, London, 1967.

The zeolite employed in the present alkylation process is normally a medium-pore size aluminosilicate zeolite. Medium pore zeolites are generally defined as those having a pore size of about 5 to about 7 Angstroms, such that the zeolite freely sorbs molecules such as n-hexane, 3-methylpentane, benzene and p-xylene. Another common definition for medium pore zeolites involves the Constraint Index test which is described in U.S. Pat. No. 4,016,218, which is incorporated herein by reference. In this case, medium pore zeolites have a Constraint Index of about 1-12, as measured on the zeolite alone without the introduction of oxide modifiers and prior to any steaming to adjust the diffusivity of the catalyst. Particular examples of suitable medium pore zeolites include ZSM-5, ZSM-11, ZSM-12, ZSM-22, ZSM-23, ZSM-35, ZSM-48, and MCM-22, with ZSM-5 and ZSM-11 being particularly preferred.

The medium pore zeolites described above are preferred for the present alkylation process since the size and shape of their pores favor the production of p-xylene over the other xylene isomers. However, although conventional forms of these zeolites have Diffusion Parameter values in excess of the 0.1-15 sec⁻¹ range referred to above, the required diffusivity can be achieved by severely steaming the catalyst so as to effect a controlled reduction in the micropore volume of the catalyst to not less than 50%, and preferably 50-90%, of that of the unsteamed catalyst. Reduction in micropore volume is derived by measuring the n-hexane adsorption capacity of the catalyst, before and after steaming, at 90° C. and 75 torr n-hexane pressure.

Steaming of the zeolite is effected at a temperature of at least about 950° C., preferably about 950 to about 1075° C., and most preferably about 1000 to about 1050° C. for about 10 minutes to about 10 hours, preferably from 30 minutes to 5 hours.

To effect the desired controlled reduction in diffusivity and micropore volume, it may be desirable to combine the zeolite, prior to steaming, with at least one oxide modifier, preferably selected from oxides of the elements of Groups IIA, IIIA, IIIB, IVA, IVB, VA and VIA of the Periodic Table (IUPAC version). Most preferably, said at least one oxide modifier is selected from oxides of boron, magnesium, calcium, lanthanum and most preferably phosphorus. In some cases, it may be desirable to combine the zeolite with more than one oxide modifier, for example a combination of phosphorus with calcium and/or magnesium, since in this way it may be possible to reduce the steaming severity needed to achieve a target diffusivity value. The total amount of oxide modifier present in the catalyst, as measured on an elemental basis, may be between about 0.05 and about 20 wt %, and preferably is between about 0.1 and about 10 wt %, based on the weight of the final catalyst.

Where the modifier includes phosphorus, incorporation of modifier into the catalyst is conveniently achieved by the methods described in U.S. Pat. Nos. 4,356,338; 5,110,776; 5,231,064; and 5,348,643, the entire disclosures of which are incorporated herein by reference. Treatment with phosphorus-containing compounds can readily be accomplished by contacting the zeolite, either alone or in combination with a binder or matrix material, with a solution of an appropriate phosphorus compound, followed by drying and calcining to convert the phosphorus to its oxide form. Contact with the phosphorus-containing compound is generally conducted at a temperature of about 25° C. and about 125° C. for a time between about 15 minutes and about 20 hours. The concentration of the phosphorus in the contact mixture may be between about 0.01 and about 30 wt %. Suitable phosphorus compounds include, but are not limited to, phosphonic, phosphinous, phosphorus and phosphoric acids, salts and esters of such acids and phosphorous halides.

After contacting with the phosphorus-containing compound, the porous crystalline material may be dried and calcined to convert the phosphorus to an oxide form. Calcination can be carried out in an inert atmosphere or in the presence of oxygen, for example, in air at a temperature of about 150 to 750° C., preferably about 300 to 500° C., for at least 1 hour, preferably 3-5 hours. Similar techniques known in the art can be used to incorporate other modifying oxides into the catalyst employed in the alkylation process.

In addition to the zeolite and modifying oxide, the catalyst employed in the alkylation process may include one or more binder or matrix materials resistant to the temperatures and other conditions employed in the process. Such materials include active and inactive materials such as clays, silica and/or metal oxides such as alumina. The latter may be either naturally occurring or in the form of gelatinous precipitates or gels including mixtures of silica and metal oxides. Use of a material which is active, tends to change the conversion and/or selectivity of the catalyst and hence is generally not preferred. Inactive materials suitably serve as diluents to control the amount of conversion in a given process so that products can be obtained economically and orderly without employing other means for controlling the rate of reaction. These materials may be incorporated into naturally occurring clays, e.g., bentonite and kaolin, to improve the crush strength of the catalyst under commercial operating conditions. Said materials, i.e., clays, oxides, etc., function as binders for the catalyst. It is desirable to provide a catalyst having good crush strength because in commercial use it is desirable to prevent the catalyst from breaking down into powder-like materials. These clay and/or oxide binders have been employed normally only for the purpose of improving the crush strength of the catalyst.

Naturally occurring clays which can be composited with the porous crystalline material include the montmorillonite and kaolin family, which families include the subbentonites, and the kaolins commonly known as Dixie, McNamee, Georgia and Florida clays or others in which the main mineral constituent is halloysite, kaolinite, dickite, nacrite, or anauxite. Such clays can be used in the raw state as originally mined or initially subjected to calcination, acid treatment or chemical modification.

In addition to the foregoing materials, the porous crystalline material can be composited with a porous matrix material such as silica-alumina, silica-magnesia, silica-zirconia, silica-thoria, silica-beryllia, silica-titania as well as ternary compositions such as silica-alumina-thoria, silica-alumina-zirconia, silica-alumina-magnesia and silica-magnesia-zirconia.

The relative proportions of porous crystalline material and inorganic oxide matrix vary widely, with the content of the former ranging from about 1 to about 90% by weight and more usually, particularly when the composite is prepared in the form of beads, in the range of about 2 to about 80 wt % of the composite. Preferably, the matrix material comprises silica or a kaolin clay.

The alkylation catalyst used in the present process may optionally be precoked. The precoking step is preferably carried out by initially utilizing the uncoked catalyst in the toluene methylation reaction, during which coke is deposited on the catalyst surface and thereafter controlled within a desired range, typically from about 1 to about 20 wt % and preferably from about 1 to about 5 wt %, by periodic regeneration by exposure to an oxygen-containing atmosphere at an elevated temperature.

One of the advantages of the catalyst described herein is its ease of regenerability. Thus, after the catalyst accumulates coke as it catalyzes the toluene methylation reaction, it can easily be regenerated by burning off a controlled amount of coke in a partial combustion atmosphere in a regenerator at temperatures in the range of from about 400 to about 700° C. The coke loading on the catalyst may thereby be reduced or substantially eliminated in the regenerator. If it is desired to maintain a given degree of coke loading, the regeneration step may be controlled such that the regenerated catalyst returning to the toluene methylation reaction zone is coke-loaded at the desired level.

The present process may suitably be carried out in fixed, moving, or fluid catalyst beds. If it is desired to continuously control the extent of coke loading, moving or fluid bed configurations are preferred. With moving or fluid bed configurations, the extent of coke loading can be controlled by varying the severity and/or the frequency of continuous oxidative regeneration in the catalyst regenerator.

Using the present process, toluene can be alkylated with methanol so as to produce para-xylene at a selectivity of about 90 wt % (based on total C8 aromatic product) at a per-pass toluene conversion of at least about 15 wt % and a trimethylbenzene production level less than 1 wt %. Thus, after removal of the unreacted feed and the small quantity of C9+ by-products, the alkylation effluent can be passed to the xylene isomerization and recovery section described below.

### Xylene Isomerization and Recovery

The C8 aromatic-containing stream recovered from the aromatic reformate fraction is combined with the C8 aromatics produced in the xylene production section described above and this combined stream is then passed to a para-xylene recovery unit to recover a para-xylene product stream and leave a para-xylene-depleted C8 stream. Typically, the para-xylene recovery unit operates by either fractional crystallization or by selective adsorption (e.g., Parex or Eluxyl).

The para-xylene-depleted C8 stream is then fed to a xylene isomerization unit where the xylenes isomers are isomerized back to their equilibrium concentrations and the ethylbenzene is converted either by cracking/disproportionation to ethane, benzene and diethylbenzene or, more preferably, by isomerization to produce further xylenes. After distillation to remove the non-C8 aromatic by-products of the isomerization process, the equilibrium C8 aromatic mixture can be recycled to the para-xylene recovery unit for recovery of further para-xylene.

In a first embodiment, where the ethylbenzene is removed by cracking/disproportionation, the para-xylene-depleted C8 stream is conveniently fed to a multi-bed reactor comprising at least a first bed containing an ethylbenzene conversion catalyst and a second bed downstream of the first bed and containing a xylene isomerization catalyst. The beds can be in the same or different reactors.

The ethylbenzene conversion catalyst typically comprises an intermediate pore size zeolite having a Constraint Index ranging from 1 to 12, a silica to alumina molar ratio of at least about 5, such as at least about 12, for example at least 20 and an alpha value of at least 5, such as 75 to 5000. Constraint Index and its method of determination are disclosed in U.S. Pat. No. 4,016,218, which is herein incorporated by reference, whereas the alpha test is described in U.S. Pat. No. 3,354,078 and in the Journal of Catalysis, Vol. 4, p. 527 (1965); Vol. 6, p. 278 (1966); and Vol. 61, p. 395 (1980), each incorporated herein by reference as to that description. The experimental conditions of the test used herein include a constant temperature of 538° C. and a variable flow rate as described in detail in the Journal of Catalysis, Vol. 61, p. 395. Higher alpha values correspond with a more active cracking catalyst.

Examples of suitable intermediate pore size zeolites include ZSM-5 (U.S. Pat. Nos. 3,702,886 and Re. 29,948); ZSM-11 (U.S. Pat. No. 3,709,979); ZSM-12 (U.S. Pat. No. 3,832,449); ZSM-22 (U.S. Pat. No. 4,556,477); ZSM-23 (U.S. Pat. No. 4,076,842); ZSM-35 (U.S. Pat. No. 4,016,245); ZSM-48 (U.S. Pat. No. 4,397,827); ZSM-57 (U.S. Pat. No. 4,046,685); and ZSM-58 (U.S. Pat. No. 4,417,780). The entire contents of the above references are incorporated by reference herein.

The zeolite employed in ethylbenzene conversion catalyst typically has a crystal size of at least 0.2 microns and exhibits an equilibrium sorption capacity for xylene, which can be either para, meta, ortho or a mixture thereof, of at least 1 gram per 100 grams of zeolite measured at 120° C. and a xylene pressure of 4.5±0.8 mm of mercury and an ortho-xylene sorption time for 30 percent of its equilibrium ortho-xylene sorption capacity of greater than 1200 minutes (at the same conditions of temperature and pressure). The sorption measurements may be carried out gravimetrically in a thermal balance. The sorption test is described in U.S. Pat. Nos. 4,117,026; 4,159,282; 5,173,461; and Re. 31,782, each of which is incorporated by reference herein.

Thus it has been found that zeolites exhibiting very high selectivity for ethylbenzene conversion while minimizing xylene loss require a very long time, that is up to or exceeding 1200 minutes to sorb ortho-xylene in an amount of 30% of their total xylene sorption capacity. For those materials, it may be more convenient to determine the sorption time for a lower extent of sorption, such as 5%, 10%, or 20% of capacity, and then to estimate the 30% sorption time by applying the following multiplication factor, F, as illustrated for 5% sorption:

t_{0.3} = F. t_{0.05}

| | Factor, F, to estimate 30% sorption |
|---|---|
| Percent sorption capacity | time t_{0.3} |
| 5 | 36 |
| 10 | 9 |
| 20 | 2.25 |

Alternatively, t_{0.3} may be calculated for other sorption times less than 30% of xylene capacity using the following relationship:

t_{0.3}=(0.3/0.x)²(t_{0.x})

where

t_{0.3} is the sorption time for 30% of total xylene capacity;

t_{0.x} is the sorption time for x % of total xylene capacity;

0.x is the fractional amount of ortho-xylene sorption to total xylene capacity

In particular, the zeolite used in the ethylbenzene conversion catalyst typically has a t_{0.3} value (in minutes) for ortho-xylene in excess of about 1200, e.g., greater than about 1500, e.g., greater than about 2000 minutes, e.g., greater than about 2500 minutes, e.g., greater than about 3000 minutes, e.g., greater than about 3600 minutes, e.g., greater than 10000 minutes, e.g., about 14760 minutes or greater.

To provide the zeolite employed in the ethylbenzene conversion catalyst with the required ortho-xylene sorption properties, the zeolite is selectivated by coking and/or by multiple organosilicon compound impregnation/calcination steps as described above for the toluene disproportionation catalyst.

The zeolite used in the ethylbenzene conversion catalyst may be self-bound (no binder) or may be composited with an inorganic oxide binder, with the zeolite content ranging from between about 1 to about 99 percent by weight and more usually in the range of about 10 to about 80 percent by weight of the dry composite, e.g., about 65% zeolite with about 35% binder. Where a binder is used, it is preferably non-acidic, such as silica. Procedures for preparing silica bound ZSM-5 are described in U.S. Pat. Nos. 4,582,815; 5,053,374; and 5,182,242, incorporated by reference herein.

In addition, the ethylbenzene conversion catalyst typically comprises from about 0.001 to about 10 percent by weight, e.g., from about 0.05 to about 5 percent by weight, e.g., from about 0.1 to about 2 percent by weight of a hydrogenation/dehydrogenation component. Examples of such components include the oxide, hydroxide, sulfide, or free metal (i.e., zero valent) forms of Group VIIIA metals (i.e., Pt, Pd, Ir, Rh, Os, Ru, Ni, Co, and Fe), Group VIIA metals (i.e., Mn, Tc, and Re), Group VIA metals (i.e., Cr, Mo, and W), Group VB metals (i.e., Sb and Bi), Group IVB metals (i.e., Sn and Pb), Group IIIB metals (i.e., Ga and In), and Group IB metals (i.e., Cu, Ag and Au). Noble metals (i.e., Pt, Pd, Ir, Rh, Os and Ru) are preferred hydrogenation/dehydrogenation components. Combinations of catalytic forms of such noble or non-noble metal, such as combinations of Pt with Sn, may be used. The metal may be in a reduced valence state, e.g., when this component is in the form of an oxide or hydroxide. The reduced valence state of this metal may be attained, in situ, during the course of a reaction, when a reducing agent, such as hydrogen, is included in the feed to the reaction.

The xylene isomerization catalyst employed in this first embodiment typically comprises an intermediate pore size zeolite, e.g., one having a Constraint Index between 1 and 12, specifically ZSM-5. The acidity of the ZSM-5 of this catalyst, expressed as the alpha value, is generally less than about 150, such as less than about 100, for example from about 5 to about 25. Such reduced alpha values can be obtained by steaming. The zeolite typically has a crystal size less than 0.2 micron and an ortho-xylene sorption time such that it requires less than 50 minutes to sorb ortho-xylene in an amount equal to 30% of its equilibrium sorption capacity for ortho-xylene at 120° C. and a xylene pressure of 4.5±0.8 mm of mercury. The xylene isomerization catalyst may used be self-bound form (no binder) or may be composited with an inorganic oxide binder, such as alumina. In addition, the xylene isomerization catalyst may contain the same hydrogenation/dehydrogenation component as the ethylbenzene conversion catalyst.

Using the catalyst system described above, ethylbenzene cracking/disproportionation and xylene isomerization are typically effected at conditions including a temperature of from about 400° F. to about 1,000° F. (204 to 538° C.), a pressure of from about 0 to about 1,000 psig (100 to 7000 kPa), a weight hourly space velocity (WHSV) of between about 0.1 and about 200 hr⁻¹, and a hydrogen, H₂ to hydrocarbon, HC, molar ratio of between about 0.1 and about 10. Alternatively, the conversion conditions may include a temperature of from about 650° F. and about 900° F. (343 to 482° C.), a pressure from about 50 and about 400 psig (446 to 2859 kPa), a WHSV of between about 3 and about 50 hr⁻¹ and a H₂ to HC molar ratio of between about 0.5 and about 5. The WHSV is based on the weight of catalyst composition, i.e., the total weight of active catalyst plus, if used, binder therefor.

In a second embodiment, where the ethylbenzene is removed by isomerization, the para-xylene-depleted C8 stream is again fed to a multi-bed reactor system but in this case the system comprises at least a first bed containing a xylene isomerization catalyst and a second bed downstream of the first bed and containing an ethylbenzene isomerization catalyst. The beds can be in the same or different reactors.

Typically, the xylene isomerization catalyst comprises an intermediate pore size molecular sieve having a Constraint Index within the approximate range of 1 to 12, such as ZSM-5 (U.S. Pat. No. 3,702,886 and Re. 29,948); ZSM-11 (U.S. Pat. No. 3,709,979); ZSM-12 (U.S. Pat. No. 3,832,449); ZSM-22 (U.S. Pat. No. 4,556,477); ZSM-23 (U.S. Pat. No. 4,076,842); ZSM-35 (U.S. Pat. No. 4,016,245); ZSM-48 (U.S. Pat. No. 4,397,827); ZSM-57 (U.S. Pat. No. 4,046,685); and ZSM-58 (U.S. Pat. No. 4,417,780). Alternatively, the xylene isomerization catalyst may comprise a molecular sieve selected from MCM-22 (described in U.S. Pat. No. 4,954,325); PSH-3 (described in U.S. Pat. No. 4,439,409); SSZ-25 (described in U.S. Pat. No. 4,826,667); MCM-36 (described in U.S. Pat. No. 5,250,277); MCM-49 (described in U.S. Pat. No. 5,236,575); and MCM-56 (described in U.S. Pat. No. 5,362,697), with MCM-49 being particularly preferred. The entire contents of the above references are incorporated by reference herein.

The xylene isomerization catalyst may also include a hydrogenation/dehydrogenation component, which may be the same material present in the second, ethylbenzene isomerization catalyst. If the same hydrogenation/dehydrogenation component is used in both catalysts, typically this component is present in a lower amount in the xylene isomerization catalyst than in the ethylbenzene isomerization catalyst. More preferably, however, to reduce its ethylbenzene conversion activity, the first catalyst composition does not contain a hydrogenation-dehydrogenation component.

In addition, it may be desirable to combine the molecular sieve of the xylene isomerization catalyst with another material resistant to the temperature and other conditions of the process. Such matrix materials include synthetic or naturally occurring substances as well as inorganic materials such as clay, silica, and/or metal oxides. The metal oxides may be naturally occurring or in the form of gelatinous precipitates or gels including mixtures of silica and metal oxides. Naturally occurring clays which can be composited with the molecular sieve include those of the montmorillonite and kaolin families, which families include the subbentonites and the kaolins commonly known as Dixie, McNamee, Georgia and Florida clays or others in which the main mineral constituent is halloysite, kaolinite, dickite, nacrite or anauxite. Such clays can be used in the raw state as originally mined or initially subjected to calcination, acid treatment or chemical modification.

In addition to the foregoing materials, the molecular sieve may be composited with a porous matrix material, such as alumina, silica-alumina, silica-magnesia, silica-zirconia, silica-thoria, silica-berylia, silica-titania, as well as ternary compounds such as silica-alumina-thoria, silica-alumina-zirconia, silica-alumina-magnesia, and silica-magnesia-zirconia. A mixture of these components could also be used. The matrix may be in the form of a cogel. The relative proportions of molecular sieve component and inorganic oxide gel matrix on an anhydrous basis may vary widely with the molecular sieve content ranging from between about 1 to about 99 percent by weight and more usually in the range of about 10 to about 80 percent by weight of the dry composite.

Typically the xylene isomerization catalyst typically has an alpha value of about 4 to about 1000, such as from about 5 to about 80, with the preferred value being inversely dependent on reactor temperature.

The second catalyst composition in this embodiment is primarily intended to isomerize the ethylbenzene in the feed selectively to para-xylene, while minimizing isomerization of the xylenes in the feed. The second catalyst composition typically comprises a molecular sieve having unidimensional 10-membered ring pores. The phrase “unidimensional 10-membered ring pores” means that the pores of the molecular sieve are defined by 10-membered rings of tetrahedrally coordinated atoms which extend essentially in one dimension so that the pores are substantially free from any intersecting pores. Examples of suitable molecular sieves having unidimensional 10-membered ring pores include SAPO-11, ZSM-23, ZSM-22, NU-87, ZSM-11, ZSM-50, ZSM-57, SAPO-41, and ZSM-48. SAPO-11 and a method of its synthesis are described in U.S. Pat. No. 4,440,871. ZSM-23 and a method of its synthesis are described in U.S. Pat. No. 4,076,842. ZSM-48 and a method of its synthesis are described in U.S. Pat. No. 4,397,827. Each of these patents is incorporated herein by reference.

The molecular sieve of the second catalyst composition typically has an alpha value of about 0.1 to about 20, for example from about 1 to about 5.

The molecular sieve used in the second catalyst composition is associated with a hydrogenation/dehydrogenation component. Examples of such components include the oxide, hydroxide, sulfide, or free metal (i.e., zerovalent) forms of Group VIII metals (i.e., Pt, Pd, Ir, Rh, Os, Ru, Ni, Co and Fe), Group VIB metals (i.e, Cr, Mo, W), Group IVA metals (i.e., Sn and Pb), Group VA metals (i.e., Sb and Bi), and Group VIIB metals (i.e., Mn, Tc and Re). Combinations of catalytic forms of such noble or non-noble metals, such as combinations of Pt with Sn, may be used. The metal is preferably in a reduced valence state. The reduced valence state of the metal may be attained, in situ, during the course of the reaction, when a reducing agent, such as hydrogen, is included in the feed to the reaction. Treatments such as coking or sulfiding may also be employed, especially at the start of a run with fresh catalyst, to modify the catalytic performance of the metal.

In one practical embodiment, the hydrogenation-dehydrogenation component is a noble metal (i.e., Pt, Pd, Ir, Rh, Os and Ru) and particularly is platinum. The amount of the hydrogenation-dehydrogenation component is suitably from about 0.001 to about 10 percent by weight, e.g., from about 0.03 to about 3 percent by weight, such as from about 0.2 to about 1 percent by weight of the total catalyst although this will, of course, vary with the nature of the component, with less of the highly active noble metals, particularly platinum, being required than of the less active base metals.

The second catalyst composition may also include a binder and/or matrix material which may be the same as, or different from, any binder and/or matrix material contained by the first catalyst composition. In particular, the binder in the second catalyst composition may be a zeolitic material such that the second catalyst composition comprises a so-called “zeolite-bound zeolite” as described in, for example, U.S. Pat. No. 6,517,807, the entire contents of which are incorporated herein by reference. Thus, the second catalyst composition may comprise a core zeolite having unidimensional 10-membered ring pores, such as ZSM-48, bound with a high silica binder which is at least partly converted to a high silica zeolite (such as ZSM-5 or ZSM-48) which at least partly covers the surface of the core zeolite. By ensuring that the zeolitic binder has a higher silica to alumina molar ratio than the core zeolite, the binder can lower the surface activity of the core zeolite and hence reduce any unwanted xylene isomerization which would otherwise occur at the surface of the core zeolite.

In general, the second catalyst composition is different from the first catalyst composition, for example by containing a different molecular sieve, having a lower alpha value and/or by containing more or a more active hydrogenation/dehydrogenation component.

The conditions employed in the xylene isomerization stage of the process of the second embodiment are not narrowly defined but generally include a temperature of from 250 to about 600° C., a pressure of from about 0 to about 500 psig (100 to 3550 kPa), a weight hourly space velocity (WHSV) of between about 0.05 and about 50 hr⁻¹, and a hydrogen, H₂, to hydrocarbon, HC, molar ratio of between about 0.05 and about 20. Typically, the xylene isomerization step is conducted in the liquid phase under conditions including a temperature of from about 250 to about 400° C., a pressure of from about 50 to about 400 psig (445 to 2870 kPa), a WHSV of between about 1 and about 10 hr⁻¹, and a H₂ to HC molar ratio of between about 1 and about 10.

The conditions used in the ethylbenzene isomerization stage are also not narrowly defined, but generally include a temperature of from about 250 to about 600° C., a pressure of from about 0 to about 500 psig (100 to 3550 kPa), a weight hourly space velocity (WHSV) of between about 0.01 and about 20 hr₋₁, and a hydrogen, H₂, to hydrocarbon, HC, molar ratio of between about 0.05 and about 20. Typically, the conditions include a temperature of from about 400 to about 500° C., a pressure of from about 50 to about 400 psig (445 to 2870 kPa), a WHSV of between about 1 and about 10 hr⁻¹, and a H₂ to HC molar ratio of between about 1 and about 10.

In general, the xylene isomerization step and the ethylbenzene isomerization step of the present process are carried out in fixed bed reaction zones containing the catalyst compositions described above. The reaction zones may be in sequential beds in a single reactor, with the ethylbenzene isomerization catalyst being located downstream of the xylene isomerization catalyst and with the feed being cascaded from the first to the second bed without intervening separation of light gases. As an alternative, the ethylbenzene isomerization catalyst and the xylene isomerization catalyst can be disposed in separate reactors which, if desired, can be operated at different process conditions, in particular with the temperature of the ethylbenzene isomerization reactor being higher than that of the xylene isomerization reactor.

### Benzene Hydrogenation

Benzene is produced in the above process during the reforming stage and potentially during the xylene production stage, especially where this involves toluene disproportionation, and during the xylene isomerization stage, especially where ethylbenzene is removed by cracking/disproportionation. In some cases, it may be desirable to hydrogenate at least part of the benzene to cyclohexane in order to consume low value hydrogen and produce a higher value, transportable product. This can be effected in known manner using a catalyst such as a Group VIII metal, for example ruthenium, palladium and/or rhodium on a porous support, at a temperature from about 50 to 250° C. and a pressure from about 1 to about 200 bar.

The invention will now be more particularly described with reference to the accompanying drawing and Example. Numerous modifications of the embodiments described below may be practiced and still be within the scope of the present invention, as set forth in the appended claims, and the embodiments should be taken as illustrative and not limiting thereof.

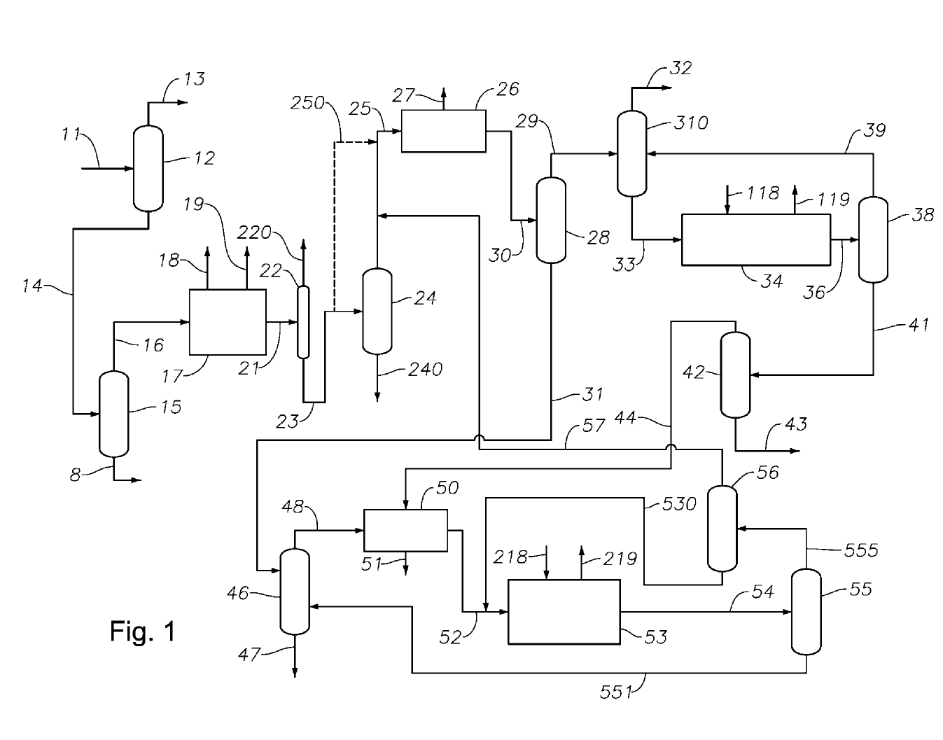

Referring to FIG. 1, a full-range naphtha is fed by line 11 to a topping tower 12 to remove a light virgin naphtha as overhead 13. The bottoms of the tower 12, in the form of a heavy virgin naphtha containing about 37 to about 54 wt % of C7 and C8 hydrocarbons, is removed from the tower 12 and fed by line 14 to a tailing tower 15, where a narrow cut naphtha containing at least 80 wt % of C7 and C8 hydrocarbons is removed as overhead and fed by line 16 to a reforming unit 17. The bottoms from tower 15 is recovered via line 8 for use as kerosene.

Passing the naphtha feed through the topping and tailing towers 12 and 15 provides a much more narrow cut for the reformer. The topping tower 12 maximizes the recovery of toluene and toluene precursors (C7 naphthenes), which rejecting benzene, C6 paraffins and C6 naphthenes. The tailing tower 15 maximizes the recovery of xylenes and xylene precursors (C8 naphthenes), while rejecting heavy (C9+) aromatic and their precursors. To minimize fractionation energy, the towers can either be heat integrated (tailing tower operating at an elevated pressure to reboil the topping tower) or the service of the towers can be combined into a single divided wall column, such modifications being within the skill of the ordinary artisan in possession of the present disclosure.

The reforming unit 17 converts at least 50 wt % of the naphthenes, but no more than 25 wt % of the paraffins, in the narrow cut naphtha feed to produce aromatic hydrocarbons, together with hydrogen and a small amount of fuel gas. The hydrogen and fuel gas by-products of the reforming process leave the unit 17 via lines 18 and 19, whereas the liquid reformate product is removed from the unit 17 via line 21 and fed to a stabilizer 22. Additional fuel gas is allowed to vent via line 220 from the reformate in stabilizer 22 before the reformate is passed via line 23 to a splitter 24 for removal of heavy (C9+) aromatic products via line 240.

After passage through the splitter 24, the C9− fraction of the reformate is fed by line 25 to an aromatics extraction unit 26. In an alternative embodiment indicated by the dotted line 250 joining lines 23 and 25 in FIG. 1, the splitter 24 is omitted and the entire reformate is passed from the stabilizer 22 to the extraction unit 26. In the aromatics extraction unit, the unconverted paraffins are separated from the reformate or the C9− fraction thereof and removed via line 27. The remaining aromatic fraction (containing benzene, toluene, xylenes, ethylbenzene and possibly C9+ aromatics) is then passed via line 30 to a fractionation column 28, where benzene and toluene are removed as overhead 29 and xylenes and heavier aromatics are removed as bottoms 31.

The overhead 29 from the column 28 is then fed to a benzene column 310, where benzene is removed via overhead line 32 and recovered, such as for purification as a product of the process or for conversion to cyclohexane. The bottoms from the benzene column 310 is composed mainly of toluene and is fed by line 33 to a toluene disproportionation unit 34 where the toluene is selectively converted to benzene and a para-rich mixture of xylene isomers, advantageously with addition of hydrogen gas 118 and with the coproduction of fuel gas taken off through line 119. The effluent from the toluene disproportionation unit 34 is fed via line 36 to a fractionation column 38, where benzene and toluene are separated from the effluent and recycled via overhead line 39 to the benzene column 310. Xylenes and heavier aromatics in the disproportionation effluent are removed from the column 38 as bottoms 41 and fed to a further fractionation column 42, where C9+ heavy by-products are separated via bottoms line 43 to leave a C8 aromatics stream, which is retrieved from the column 42 via overhead line 44.

The bottoms 31 from the column 28 is fed to a xylene rerun column 46 where C9+ aromatics are separated and removed via bottoms line 47 to leave a further C8 aromatics stream, which is removed from the column 46 by overhead line 48. The C8 aromatics streams in lines 44 and 48 are fed to a para-xylene recovery unit 50, where a para-xylene product stream is recovered via 51. The para-xylene-depleted C8 stream remaining after recovery of the para-xylene product stream is fed by line 52 to a xylene isomerization unit 53 where the xylenes and ethylbenzene in the para-xylene-depleted stream are isomerized back towards an equilibrium mixture, advantageously in the presence of hydrogen gas provided by line 218 and with the coproduction of fuel gas taken off through line 219. The effluent from the xylene isomerization unit 53 is fed by line 54 to a fractionation column 55, where the C8+ components are separated as bottoms and recycled via line 551 to the xylene rerun column 46. The overhead from the column 55 is composed mainly of benzene, toluene and naphthene intermediates and is passed via line 555 to a further fractionation column 56, where the benzene and toluene are separated and fed by line 57 to line 25 (or, not shown, optionally to line 250, if present) and then to the extraction unit 26, while the naphthenes are recycled via line 530 to the xylene isomerization unit 53, which may be by direct connection, not shown, or, as shown, via line 52.

In a modification (not shown) of the embodiment shown in FIG. 1, one or more of the pairs of columns 12 and 15, 28 and 310 and 38 and 42 is replaced by a single divided wall column. Another modification (not shown) of the embodiment shown in FIG. 1, columns 55 and 56 can be replaced by a single column or divided wall column. A combination of these modifications can also be adopted.

### Example 1

Table 1 provides the estimated material balance for the process shown in FIG. 1, in which the xylene isomerization unit 53 converts ethylbenzene by isomerization to xylenes and all rates are listed in kilotons per annum.

TABLE 1

| | Stream | Rate |
|---|---|---|
| Refinery Streams | Full-range Naphtha Feed | 5742 |
| | Light Virgin Naphtha | 2259 |
| | C7/C8 Cut | 3280 |
| | Heavy Virgin Naphtha | 202 |
| | Reformate | 3159 |
| Main Product Streams | Raffinate | 1718 |
| | Benzene | 312 |
| | Para-xylene | 960 |
| By-Product Streams | H₂ | 67 |
| | Fuel Gas | 47 |
| | LPG | 43 |
| | C9+ Aromatics | 106 |
| | Light H/C | 37 |

It will be seen that using the process of FIG. 1 to produce 960 kT/a of para-xylene, the total light gas make is 157 kilotons/annum or about 3% of the total hydrocarbon feed converted in the process.

By way of comparison, Table 2 provides the estimated material balance for a conventional aromatics production process, in which a high severity reformer is used to feed a C9+ transalkylation unit and xylene isomerization unit which converts ethylbenzene by cracking to benzene and ethane. Again all rates are listed in kilotons per annum.

TABLE 2

| | Stream | Rate |
|---|---|---|
| Refinery Streams | Full-range Naphtha Feed | 2633 |
| | Light Virgin Naphtha | 584 |
| | Heavy Virgin Naphtha | 2049 |
| | Reformate | 1829 |
| Main Product Streams | Raffinate | 385 |
| | Benzene | 357 |
| | Para-xylene | 960 |
| By-Product Streams | H₂ | 62 |
| | Fuel Gas | 46 |
| | LPG | 113 |
| | C9+ Aromatics | 10 |
| | Light H/C | 65 |

It will be seen that using the conventional aromatics production process (Table 2) to produce a similar amount of para-xylene as the process of the invention (Table 1), there is a significant downgrading of higher molecular weight molecules to gas, the former having higher value in general than the latter, particularly in regions of the world where light gases have little or no utility. By way of example, the total light gas make in the conventional process shown in Table 2 is 221 kilotons/annum or almost 50% more than that obtained in the process of Example 1, and other refinery streams obtainable by the process of the invention, such as light virgin naphtha, are significantly increased in the process of the invention as compared with the conventional process.

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

1. A process for producing para-xylene, the process comprising:
(a) reforming a naphtha feed under reforming conditions effective to convert at least 50 wt % of the naphthenes in the naphtha feed to aromatics, but to convert no more than 25 wt % of the paraffins in the naphtha feed, and thereby produce a reforming effluent;
(b) removing at least a first stream containing benzene and/or toluene and a second stream containing C8 aromatics from the reforming effluent;
(c) feeding at least part of the benzene and/or toluene from the first stream to a xylene production unit under conditions effective to convert benzene and/or toluene to xylenes;
(d) feeding at least part of the C8 aromatics from the second stream and at least part of the xylenes produced in (c) to a para-xylene recovery unit to recover a para-xylene product stream and leave a para-xylene-depleted C8 stream;
(e) feeding at least part of para-xylene-depleted C8 stream to a xylene isomerization unit effective to isomerize xylenes in said stream back towards an equilibrium mixture of xylenes and thereby produce an isomerization effluent; and
(f) recycling the isomerization effluent to the para-xylene extraction unit.

2. The process of claim 1, wherein the naphtha feed contains at least 25 wt % of C7 and C8 hydrocarbons.

3. The process of claim 2, wherein the naphtha feed contains at least 35 wt % of C7 and C8 hydrocarbons.

4. The process of claim 2, wherein the naphtha feed contains at least 80 wt % of C7 and C8 hydrocarbons.

5. The process of claim 1, wherein the reforming (a) is conducted in one or more fixed bed reforming units.

6. The process of claim 1, wherein said removing (b) comprises a solvent extraction and/or extractive distillation to separate the reforming effluent into an aromatics fraction and a non-aromatics fraction.

7. The process of claim 6, further comprising separating benzene from said aromatics fraction.

8. The process of claim 7, further comprising reacting at least part of the benzene separated from said aromatics fraction with hydrogen produced by said reforming (a) to convert said benzene to cyclohexane.

9. The process of claim 1, wherein said xylene production unit effects disproportionation of toluene to produce benzene and xylenes.

10. The process of claim 9, further comprising reacting at least part of the benzene produced in the toluene disproportion unit with hydrogen produced by said reforming (a) to convert said benzene to cyclohexane.

11. The process of claim 1, wherein said xylene production unit effects alkylation of benzene and/or toluene with methanol to produce xylenes.

12. The process of claim 1, wherein the xylene isomerization unit is effective to convert ethylbenzene in said para-xylene-depleted C8 stream to xylenes.

13. The process of claim 1, wherein no more than 10 wt % of said feed is converted to hydrocarbons having 4 or less carbon atoms in steps (a), (c) and (e) combined.

14. A para-xylene production plant comprising:
(a) a first separation system for removing C6− hydrocarbons and C9+ hydrocarbons from a C₅ to C₁₂ hydrocarbon fraction to produce a naphtha feed;
(b) at least one reforming unit for converting at least 50 wt % of the naphthenes in the naphtha feed to aromatics, but to convert no more than 25 wt % of the paraffins in the naphtha feed, and thereby produce a reforming effluent;
(c) a second separation system for separating the reforming effluent into an aromatics fraction and a non-aromatics fraction;
(d) a third separation system for separating the aromatics fraction into a first stream containing benzene and/or toluene and a second C8 aromatic-containing stream;
(e) a xylene production unit for converting at least part of the benzene and/or toluene in the first stream to xylenes;
(f) a fourth separation system for selectively recovering para-xylene from the second C8 aromatic-containing stream and the xylenes produced in said xylene production unit to leave a para-xylene-depleted C8 stream;
(g) a xylene isomerization unit effective to isomerize xylenes in said para-xylene-depleted C8 stream back towards an equilibrium mixture of xylenes and to isomerize ethylbenzene in said stream to xylenes and thereby produce an isomerization effluent; and
(h) means for recycling said isomerization effluent to the fourth separation system.

15. The plant of claim 14, wherein the or each reforming unit comprises a fixed bed reactor.

16. The plant of claim 14, wherein the second separation system comprises a liquid-liquid extraction unit.

17. The plant of claim 14, wherein the fourth separation system comprises a selective crystallization unit or a selective adsorption unit.

18. The plant of claim 14, further comprising a hydrogenation unit for converting at least part of the benzene produced in the toluene disproportion unit and/or at least part of the benzene in said benzene-containing stream to cyclohexane.
